(19) United States
(12) Patent Application Publication
(10) Pub. No.: US 2003/0082121 A1
(43) Pub. Date: May 1, 2003

(54) Color changing nail polish

(76) Inventors: Benny Borsakian, Encino, CA (US); Janel Faraci, Encino, CA (US)

Correspondence Address: Norton R. Townsley, 100 Corporate Pointe, Suite 330, Culver City, CA, 90230, US

(21) Appl. No.: 10/029,498

(22) Filed: Dec. 19, 2001

Related U.S. Application Data
(60) Provisional application No. 60/305,595, filed on Jul. 13, 2001.

(51) Int. Cl. A61K 7/04

(52) U.S. Cl. 424/61

(57) ABSTRACT

A nail polish composition comprising a nail polish base into which is mixed a colorant a temperature sensitive colorant and a UV photochromic powder. The colorant, temperature sensitive colorant and UV photochromic powder imparting, after the nail polish composition is applied to a human nail and allowed to dry, a first color when the human nail is at normal body temperature, a second color when the temperature of the human nail is above normal body temperature, and a third color when the nail polish composition is exposed to UV radiation.

### CROSS REFERENCE

The Applicants claim the benefit of their Provisional Application No. 60/305,595, filed 07/19/2001.

BACKGROUND OF THE INVENTION

The present invention relates to nail polish and more particularly to a nail polish that has the ability of changing color upon exposure to heat and ultraviolet radiation, for example sunlight.

Nail polish comprises a class of products regularly used by women as part of their beauty care. Enamel nail polishes are commercially available indifferent colors and shades. Women use this beauty accessory to match their wardrobe, jewelry and makeup. However, to date there is no nail polish available in the market that can change color with changes in body heat of the wearer or upon exposure to sunlight (ultraviolet radiation).

Nail polish manufacturers have focused their work in producing a fast drying nail coat as shown by the numerous patents in this area. For example, U.S. Pat. No. 5,435,994 or U.S. Pat. No. 5,456,905. A photocurable nail lacquer has been developed by Rosenberg (see U.S. Pat. Nos. 3,896,014 and 3,928,113). The purpose of the idea was to develop a water removable nail coat instead of the usual acetone based remover. In U.S. Pat. No. 5,435,994, Valenty describes a photoreactive nail polish coating composition that cures quickly upon exposure to low levels of ultraviolet radiation. The coating consists of a polymer formed from nitrocellulose, a photoreactive monomer, a photoinitiator, and a reaction inhibitor resulting in a product compatible with commercially available nail polish of any color. The coating is removable by commercially available acetone based polish removers. The nail polish dries in few minutes by the use of a small dose of UV radiation. In a subsequent patent, U.S. Pat. No. 5,456,905, Valenti describes a photoreactive nail polish that dries quickly upon exposure to natural light.

In U.S. Pat. No. 5,591,255, Small and Highberger teach a thermochromic ink formulation that is sensitive to temperature. In particular, they show a nail lacquer having the property of changing color with change of temperature. However, thermochromic dyes go from a color to no color therefore a color enamel has to be initially applied to produce the desired color change effect.

In U.S. Pat. No. 5,730,961, Goudjil describes what he calls a metamorphic nail polish. This is capable of changing from clear to any visible color (violet, red, blue, yellow etc.) in presence of sunlight or any ultraviolet radiation source. The nail polish will go back to colorless as soon as it is removed from the sunlight or the UV source. The active chemical is a photochromic substance such as spiropyrans or spiroxazines. The photochromic compound is added to any clear nail polish to form a composition sensitive to UV radiation.

None of the references found show a nail polish capable of changing color as the ambient temperature changes and upon exposure to ultraviolet light (e.g., sunlight).

Development of a nail polish which can change color as the temperature changes and upon exposure to ultraviolet radiation represents a great improvement in the field of nail polishes and satisfies a long felt need of the nail polish wearers.

### SUMMARY OF THE INVENTION

This invention is a nail polish which can change color as the temperature of the wearer changes and upon exposure to ultraviolet radiation. The nail polish comprises: a film forming chemical, a plasticizer, a solvent, a colorant, a temperature sensitive colorant, a UV absorber, an additive, a viscosity adjuster, and a photochromic powder. After the nail polish is applied to a nail and allowed to dry the combination of the colorant, temperature sensitive colorant and photochromic powder allow the nail polish to exhibit a first color when the nail is at normal body temperature, a second color when the temperature of the nail is above normal body temperature, and a third color when the nail polish composition is exposed to UV radiation.

An appreciation of the other aims and objectives of the present invention and an understanding of it may be achieved by referring to the accompanying drawings and description of a preferred embodiment.

### DETAILED DESCRIPTION OF THE PREFERRED EMBODIMENT

This invention comprises: a mixture of film forming chemicals, a mixture of plasticizers, a mixture of solvents, a mixture of colorants, a mixture of temperature sensitive colorants, a UV absorber, an additive, a viscosity adjuster, and a mixture of photochromic powders. The preferred ingredients and their composition ranges for this invention are provided in Table I.

TABLE I

| Item | | | Range |
|---|---|---|---|
| Number | Ingredient | Function | % by weight |
| 1 | Ethyl Acetate | Solvent | 21.00-38.00 |
| 2 | Butyl Acetate | Solvent | 16.00-24.00 |
| 3 | Nitrocellulose | Film Former | 6.00-10.00 |
| 4 | Sucrose Acetate Isobutyrate | Plasticizer | 1.00-7.00 |
| 5 | Isopropanol | Solvent | 1.00-4.00 |
| 6 | Hydroxyethyl Acrylate-Butyl Acrylate- | Film Former | 1.00-3.00 |
| | N-methoxyethyl Acrylate Copolymer Solution (1) | | |
| 7 | Dibutyl Phthalate | Plasticizer | 1.00-3.00 |
| 8 | Benzyldimethlsterylarnmonirn Hectorite (1) | Viscosity Agent | 1.00-2.00 |
| 9 | DL-Camphor (1) | Plasticizer | 0.25-1.00 |
| 10 | Bismuth Oxychloride | Pearlescent Colorant | 0.00-0.50 |
| 11 | Yellow No.4 (1) | Colorant | 0.00-2.50 |
| 12 | Titanium Dioxide | Colorant | 0.00-3.00 |
| 13 | Benzophenone (1) | UV Absorber | 0.10-1.00 |
| 14 | Iron Blue (1) | Pigment-Colorant | 0.00-3.00 |
| 15 | Citric Acid | Additive | 0.10-2.00 |
| 16 | Black Iron Oxide (1) | Pigment-Colorant | 0.00-2.00 |
| 17 | Red #6 (1) | Pigment-Colorant | 0.00-1.00 |
| 18 | Red #7 (1) | Pigment-Colorant | 0.00-3.00 |
| 19 | Mica (3) | Pearlescent Colorant | 0.00-5.00 |
| 20 | Aluminum Powder (3) | Pearlescent Colorant | 0.00-1.00 |
| 21 | Red Iron Oxide (3) | Pigment-Colorant | 0.00-0.50 |
| 22 | Brilliant Rose (2) | Temperature Sensitive | 0.00-2.00 |
| | | Colorant | |
| 23 | Brilliant Green (2) | Temperature Sensitive | 0.00-2.00 |
| | | Colorant | |
| 24 | Brown (2) | Temperature Sensitive | 0.00-1.00 |
| | | Colorant | |
| 25 | Fast Black (2) | Temperature Sensitive | 0.00-0.50 |
| | | Colorant | |
| 26 | Fast Blue (2) | Temperature Sensitive | 0.00-5.00 |
| | | Colorant | |
| 27 | Gold Orange (2) | Temperature Sensitive | 0.00-2.00 |
| | | Colorant | |
| 28 | Magenta (2) | Temperature Sensitive | 0.00-8.00 |
| | | Colorant | |
| 29 | Yellow (2) | Temperature Sensitive | 0.00-5.00 |
| | | Colorant | |
| 30 | Pink (2) | Temperature Sensitive | 0.00-3.00 |
| | | Colorant | |
| 31 | Turquoise Blue (2) | Temperature Sensitive | 0.00-3.50 |
| | | Colorant | |
| 32 | Vermillion (2) | Temperature Sensitive | 0.00-0.50 |
| | | Colorant | |
| 33 | Acrylic Resin (2) | Plasticizer | 0.25-4.00 |
| 34 | Petroleum Resin (2) | Plasticizer | 0.25-3.00 |
| 35 | Aromatic Solvent (2) | Solvent | 0.25-3.00 |
| 36 | Red Powder (2) | UV Photochromic | 0.00-3.00 |
| | | Powder | |
| 37 | Purple Powder (2) | UV Photochromic | 0.00-0.60 |
| | | Powder | |
| 38 | Blue Powder (2) | uv Photochromic | 0.00-0.50 |
| | | Powoer | |
| 39 | Yellow Powder (2) | UV Photochromic | 0.00-1.00 |
| | | Powder | |
| 40 | Guanine (3) | Natural Pearlescent | 0.00-0.50 |
| | | colorant | |
| 41 | Chromicolor (2) | Temperature Sensitive | 0.00-2.00 |
| | | Colorant | |

(1) Available from Tevco, Inc., South Plainfield, NJ
(2) Available from Matsui Shikso Chemical Co., Kyoto, Japan. US office: Matsui International Co, Inc., Gardena, CA
(3) Available from Merck KgaA, Darmstadt, Germany. US office: Rona, E.M. Industries, Hawthorne, NY

Some of the ingredients come premixed. For example, items 33, 34, 35 and 41 are supplied as Chromicolor Gravure Ink NC Base by Matsui Shikso Chemical Co., Kyoto, Japan. US office: Matsui International Co, Inc., Gardena, Calif. Also, items 1-9, 1-19 and 22-31 can be procured premixed.

The film formers, plasticizers, solvents and other additives are designed to create a nail polish base that can be brushed onto a human nail to form a coating when dried. The colorants are of several varieties, namely pearlescent colorants, and pigment colorants. These are added to the base to impart a first color. This is the color that the nail polish exhibits when it is first applied. The temperature sensitive colorants change color as ambient temperature increases. These impart a second color to the nail polish as the temperature of the polish, actually of the wearer of the polish, increases. Only a small temperature change of about 0.5° F. is sufficient to effectuate this color change. The UV photochromic powders have the property of exhibiting different colors under visible and UV light. They impart a different color to the nail polish when it is exposed to UV light, as for example in broad sunlight.

Specific concentrations of ingredients, within the above referenced ranges, particularly the colored ingredients, are selected and mixed by standard procedures to produce a nail polish with the desired properties. After the nail polish is applied to a nail and allowed to dry the combination of the colorant, temperature sensitive colorant and photochromic powder allow the nail polish to exhibit a first color when the nail is at normal body temperature, a second color when the temperature of the nail is above normal body temperature, and a third color when the nail polish composition is exposed to UV radiation. Above normal body temperatures result, for example, when the individual exercises or exposes her hand to hot water. In other words a temperature rise of about 0.5° F. is sufficient to achieve the color change. When body temperature again falls to normal, the polish regains the first color.

### EXAMPLES

Nail polishes were mixed according to the formulae in Table II.

TABLE II

| Item | | Formula | | | | | |
|---|---|---|---|---|---|---|---|
| Number | Ingredient | A | B | C | D | E | F |
| 1 | Ethyl Acetate | 23.20% | 26.05% | 28.93% | 21.98% | 22.87% | 29.79% |
| 2 | Butly Acetate | 18.90% | 20.90% | 20.84% | 16.64% | 18.90% | 18.90% |
| 3 | Nitrocellulose | 7.11% | 7.11% | 7.36% | 6.11% | 7.11% | 7.11% |
| 4 | Sucrose Acetate Isobutyrate | 2.00% | 2.00% | 1.99% | 2.00% | 2.00% | 2.00% |
| 5 | Isopropanol | 5.80% | 5.80% | 4.86% | 3.66% | 5.80% | 5.80% |
| 6 | Hydroxyethyl Acrylate-Butyl | 2.50% | 2.50% | 2.39% | 2.50% | 2.50% | 2.50% |
| | Acrylate-N-methoxyethyl Acrylate | | | | | | |
| | Copolymer Solution | | | | | | |
| 7 | Dibutyl Phthalate | 1.85% | 1.85% | 1.66% | 1.00% | 1.85% | 1.85% |
| 8 | Benzyldimehtylsterylammonium | 1.25% | 1.25% | 1.24% | 1.25% | 1.25% | 1.25% |
| | Hectorite | | | | | | |
| 9 | DL-Camphor | 0.78% | 0.78% | 0.75% | 0.78% | 0.78% | 0.78% |
| 10 | Bismuth Oxychloride | 0.25% | 0.25% | 0.23% | 0.25% | 0.25% | 0.25% |
| 11 | Yellow No. 4 | 0.10% | 0.10% | 0.10% | 0.10% | 2.25% | 0.32% |
| 12 | Titanium Dioxide | 2.50% | 2.50% | 0.65% | 2.50% | 2.50% | 2.50% |
| 13 | Benzophenone | 0.65% | 0.65% | 0.78% | 0.65% | 0.65% | 0.65% |
| 14 | Iron Blue | 1.20% | 1.20% | | 2.00% | 1.20% | 2.11% |
| 15 | Citric Acid | 2.00% | 2.00% | 2.00% | 2.00% | 2.00% | 1.98% |
| 16 | Black Iron Oxide | 1.50% | 1.50% | | 1.48% | 1.50% | |
| 17 | Red # 6 | 0.34% | 0.34% | 1.00% | 0.34% | 0.34% | 0.34% |
| 18 | Red # 7 | | | 1.22% | 2.50% | | 1.00% |
| 19 | Mica | 2.85% | 1.00% | 5.00% | 2.14% | 2.96% | 3.00% |
| 20 | Aluminum Powder | | | 0.09% | | | 1.00% |
| 21 | Red Iron Oxide | | | 0.36% | | | 0.13% |
| 22 | Brilliant Rose | | | 2.00% | | | 0.85% |
| 23 | Brilliant Green | 0.05% | 0.05% | 0.01% | 0.05% | | |
| 24 | Brown | 0.90% | 0.90% | 0.10% | 0.90% | 0.90% | |
| 25 | Fast Black | | | | | | |
| 26 | Fast Blue | 5.00% | 2.00% | | 3.37% | | |
| 27 | Gold Orange | | | 2.00% | | | 0.85% |
| 28 | Magenta | 8.00% | 8.00% | 0.10% | 8.00% | 8.00% | 1.00% |
| 29 | Yellow | | | 0.22% | | 3.12% | |
| 30 | Pink | | | 1.89% | 2.89% | | 2.00% |
| 31 | Turquoise Blue | | | | 3.50% | | |
| 32 | Vermillion | | | | 0.14% | | |
| 33 | Acrylic Resin | 3.30% | 3.30% | 2.89% | 3.30% | 3.30% | 3.30% |
| 34 | Petroleum Resin | 2.85% | 2.85% | 2.98% | 2.85% | 2.85% | 2.85% |
| 35 | Aromatic Solvent | 2.16% | 2.16% | 1.97% | 2.16% | 2.16% | 2.16% |
| 36 | Red Powder | 0.10% | 0.10% | 3.00% | 1.07% | 0.10% | 0.10% |
| 37 | Purple Powder | 0.52% | 0.52% | 0.01% | 0.52% | 0.52% | 0.52% |
| 38 | Blue Powder | | | | 0.13% | | |
| 39 | Yellow Powder | 0.98% | 0.98% | | | 0.98% | 1.00% |
| 40 | Guanine | 0.11% | 0.11% | 0.13% | | 0.11% | 0.11% |
| 41 | Chromicolor | 1.25% | 1.25% | 1.25% | 1.25% | 1.25% | 2.00% |
| | | 100.00% | 100.00% | 100.00% | 100.01% | 100.00% | 100.00% |

Each formula was applied to a human nail and allowed to dry. The colors exhibited by the different formulae are shown in Table III.

TABLE III

| | Formula | | | | | |
|---|---|---|---|---|---|---|
| | A | B | C | D | E | F |
| First color | Mauve | Medium | Purple | Purple | Medium | Neon |
| (normal body | | Blue | | | Pink | Pink |
| temperature) | | | | | | |
| Second color | Silver | Light | Light | Silver | White | Purple |
| (above normal | Gray | Blue | Blue | | | |
| body | | | | | | |
| temperature) | | | | | | |
| Third color | Mustard | Green | Green | Dark | Yellow | Red |
| (UV exposure) | Gold | Gold | Gold | Gold | | |

The color changing nail polish has been described with reference to a particular embodiment. Other modifications and enhancements can be made without departing from the spirit and scope of the claims that follow.

What is claimed is:

1. A nail polish composition comprising:
a. a mixture of chemicals designed to form a coating for a human nail;
b. a colorant;
c. a temperature sensitive colorant; and
d. a UV photochromic powder; said colorant, temperature sensitive colorant and UV photochromic powder imparting, after said nail polish composition is applied to a human nail and allowed to dry, a first color when said human nail is at normal body temperature, a second color when the temperature of said human nail is above normal body temperature, and a third color when said nail polish composition is exposed to UV radiation.

2. A nail polish composition comprising:
a. a film forming chemical;
b. a plasticizer;
c. a solvent;
d. a colorant;
e. a temperature sensitive colorant;
f. a UV absorber;
g. an additive;
h. a viscosity adjuster; and
i. a UV photochromic powder; said colorant, temperature sensitive colorant and UV photochromic powder imparting, after said nail polish composition is applied to a human nail and allowed to dry, a first color when said human nail is at normal body temperature, a second color when the temperature of said human nail is above normal body temperature, and a third color when said nail polish composition is exposed to UV radiation.

3. A composition as claimed in claim 2 in which said film forming chemical is selected from the group consisting of nitrocellulose, hydroxyethyl acrylate, butyl acrylate, n-methoxyethyl acrylate, and their mixtures.

4. A composition as claimed in claim 2 in which said plasticizer is selected from the group consisting of sucrose acetate isobutyrate, dibutyl phthalate, DL-camphor, acrylic resin, petroleum resin and their mixtures.

5. A composition as claimed in claim 2 in which said solvent is selected from the group consisting of ethyl acetate, butyl acetate, isopropanol, aromatic solvent and their mixtures.

6. A composition as claimed in claim 2 in which said colorant is selected from the group consisting of yellow no.4, titanium dioxide, guanine, bismuth oxychloride, mica, aluminum powder, iron blue, black oxide of iron, red #6, red #7, red oxide of iron, and their mixtures.

7. A composition as claimed in claim 2 in which said temperature sensitive colorant is selected from the group consisting of brilliant rose, brilliant green, brown, fast black, fast blue, gold orange, magenta, yellow, pink, turquoise blue, vermillion, Chromicolor Gravure Ink NC Base, and their mixtures.

8. A composition as claimed in claim 2 in which said UV absorber is benzophenone.

9. A composition as clamed in claim 2 in which said additive is citric acid.

10. A composition as claimed in claim 2 in which said viscosity adjuster is benzyldimethlsterylarnmonim hectorite

11. A composition as claimed in claim 2 in which said UV photochromic powder is selected from the group consisting of red, purple, yellow, blue, and their mixtures.

12. A nail polish composition comprising:

| | | |
|---|---|---|
| 21.00-38.00 | % by weight | Ethyl Acetate |
| 16.00-24.00 | % by weight | Butyl Acetate |
| 6.00-10.00 | % by weight | Nitrocellulose |
| 1.00-7.00 | % by weight | Sucrose Acetate Isobutyrate |
| 1.00-4.00 | % by weight | Isopropanol |
| | | Hydroxyethyl Acrylate-Butyl Acrylate- |
| 1.00-3.00 | % by weight | N-methoxyethyl Acrylate Copolymer |
| | | Solution |
| 1.00-3.00 | % by weight | Dibutyl Phthalate |
| 1.00-2.00 | % by weight | Benzyldimethlsterylarnmonirn Hectorite |
| 0.25-1.00 | % by weight | DL-Camphor |
| 0.00-0.50 | % by weight | Bismuth Oxychloride |
| 0.00-2.50 | % by weight | Yellow No.4 Colorant |
| 0.00-3.00 | % by weight | Titanium Dioxide |
| 0.10-1.00 | % by weight | Benzophenone |
| 0.00-3.00 | % by weight | Iron Blue Colorant |
| 0.10-2.00 | % by weight | Citric Acid |
| 0.00-2.00 | % by weight | Black Oxide Iron Colorant |
| 0.00-1.00 | % by weight | Red#6 Colorant |
| 0.00-3.00 | % by weight | Red#7 Colorant |
| 0.00-5.00 | % by weight | Mica |
| 0.00-1.00 | % by weight | Aluminum Powder |
| 0.00-0.50 | % by weight | Red Oxide of Iron Colorant |
| 0.00-2.00 | % by weight | Brilliant Rose Temperature Sensitive |
| | | Colorant |
| 0.00-2.00 | % by weight | Brilliant Green Temperature Sensitive |
| | | Colorant |
| 0.00-1.00 | % by weight | Brown Temperature Sensitive Colorant |
| 0.00-0.50 | % by weight | Fast Black Temperature Sensitive Colorant |
| 0.00-5.00 | % by weight | Fast Blue Temperature Sensitive Colorant |
| 0.00-2.00 | % by weight | Gold Orange Temperature Sensitive Colorant |
| 0.00-8.00 | % by weight | Magenta Temperature Sensitive Colorant |
| 0.00-5.00 | % by weight | Yellow Temperature Sensitive Colorant |
| 0.00-3.00 | % by weight | Pink Temperature Sensitive Colorant |
| 0.00-3.50 | % by weight | Turquoise Blue Temperature Sensitive |
| | | Colorant |
| 0.00-0.50 | % by weight | Vermillion Temperature Sensitive Colorant |
| 0.25-4.00 | % by weight | Acrylic Resin |
| 0.25-3.00 | % by weight | Petroleum Resin |
| 0.25-3.00 | % by weight | Aromatic Solvent |
| 0.00-3.00 | % by weight | Red UV Photochromic Powder |
| 0.00-0.60 | % by weight | Purple Photochromic Powder |
| 0.00-0.50 | % by weight | Blue Photochromic Powder |
| 0.00-1.00 | % by weight | Yellow Photochromic Powder |
| 0.00-0.50 | % by weight | Guanine |
| 0.00-2.00 | % by weight | Chromicolor |

13. A method of fabricating a nail polish composition comprising the step of mixing together a film forming chemical, a plasticizer, a solvent, a colorant, a temperature sensitive colorant, a UV absorber, an additive, a viscosity adjuster, and a UV photochromic powder; said colorant, temperature sensitive colorant and UV photochromic powder imparting, after said nail polish composition is applied to a human nail and allowed to dry, a first color when said human nail is at normal body temperature, a second color when the temperature of said human nail is above normal body temperature, and a third color when said nail polish composition is exposed to UV radiation.

14. A composition as claimed in claim 13 in which said film forming chemical is selected from the group consisting of nitrocellulose, hydroxyethyl acrylate, butyl acrylate, n-methoxyethyl acrylate, and their mixtures.

15. A composition as claimed in claim 13 in which said plasticizer is selected from the group consisting of sucrose acetate isobutyrate, dibutyl phthalate, DL-camphor, acrylic resin, petroleum resin and their mixtures.

16. A composition as claimed in claim 13 in which said solvent is selected from the group consisting of ethyl acetate, butyl acetate, isopropanol, aromatic solvent and their mixtures.

17. A composition as claimed in claim 13 in which said colorant is selected from the group consisting of yellow no.4, titanium dioxide, guanine, bismuth oxychloride, mica, aluminum powder, iron blue, black oxide of iron, red #6, red #7, red oxide of iron, and their mixtures.

18. A composition as claimed in claim 13 in which said temperature sensitive colorant is selected from the group consisting of brilliant rose, brilliant green, brown, fast black, fast blue, gold orange, magenta, yellow, pink, turquoise blue, vermillion, Chromicolor Gravure Ink NC Base, and their mixtures.

19. A composition as claimed in claim 13 in which said UV absorber is benzophenone.

20. A composition as clamed in claim 13 in which said additive is citric acid.

21. A composition as claimed in claim 13 in which said viscosity adjuster is benzyldimethlsterylarnmonirn hectorite

22. A composition as claimed in claim 13 in which said UV photochromic powder is selected from the group consisting of red, purple, yellow, blue, and their mixtures.
